(19) United States
(12) Patent Application Publication
(10) Pub. No.: US 2026/0060740 A1
(43) Pub. Date: Mar. 5, 2026

(54) ELECTROSURGICAL APPARATUS WITH CURRENT SENSOR FOR TREATMENT CURRENT

(71) Applicant: Erbe Elektromedizin GmbH, Tuebingen (DE)

(72) Inventors: Erich WERNER, Wannweil (DE); Mike Puggé, Bempflingen (DE)

(21) Appl. No.: 19/315,947

(22) Filed: Sep. 2, 2025

(30) Foreign Application Priority Data
Sep. 5, 2024 (EP) 24198682.7

(51) Int. Cl. A61B 18/12; A61B 17/00; A61B 18/00

(52) U.S. Cl. CPC A61B 18/1206; A61B 2017/00862; A61B 2018/00083; A61B 2018/00107; A61B 2018/00833

(57) ABSTRACT

A ring-shaped magnetic circuit is provided for a current sensor of an apparatus for supplying an instrument, wherein two lines supplying the instrument and a neutral electrode or the instrument pass through the magnetic circuit. The coatings of the two lines comprise sufficient thickness with regard to their dielectric strength to the voltage load of lines. The thickness is limited so that an insulation body fits between the two lines consisting of a nonpolar insulation material with high insulation capabilities, similar to the coatings, such as a plastic with a low loss angle. The insulation body reduces partial discharges in the area of the current sensor, which would otherwise result in electromagnetic emissions and thus distortions of the signals created by the current sensor.

### CROSS REFERENCE TO RELATED APPLICATIONS

This application claims the benefit of European Patent Application No. 24198682.7, filed Sep. 5, 2024, which is incorporated herein by reference in its entirety.

### TECHNICAL FIELD

The invention refers to an electrosurgical apparatus having a generator for a supply of an electrosurgical instrument with which interventions on a human or animal patient can be carried out.

### BACKGROUND

For treatment of human or animal bodies monopolar or bipolar instruments are known that are supplied with high-frequency current (RF current) under considerable voltage. The voltage can range from multiple 10 V up to multiple 1000 V. Thereby it is frequently required to measure the current supplied to the instrument precisely and also with high time resolution in order to control the generator accordingly.

A current sensor is known from practice comprising a magnetic circuit through which the line leading to the instrument as well as the line leading back from the instrument of the patient are passing. A sensor coil also provided on the magnetic circuit or other means for detection of a magnetic field provide a signal that characterizes the detected current.

Between the lines a considerable voltage can be present depending on the treatment type which can result in partial discharges within the insulation of the lines guided in close proximity to each other, particularly in case of unfavorable use conditions, for example use conditions in high altitude, at low air pressure, at high air humidity, etc. Such partial discharges between the two lines guiding high-frequency current can cause disturbances that result in falsified measurements or even error messages of the connected sensor technology. This happens also if they are so low that they are harmless with regard to the dielectric strength of the insulation.

An enlargement of the insulation thickness of the individual lines is obstructed by the constricted space conditions in respective current sensors.

### SUMMARY

Starting therefrom one object of the invention is to provide an apparatus for supply of an electrosurgical instrument that allows a reliable detection of the current supply to the instrument also under adverse use conditions.

An apparatus according to the invention comprises a generator having an output circuit that is connected with apparatus outputs via lines. These lines pass through a central opening of a magnetic circuit of a current sensor, wherein an insulation body is arranged between the two lines. This insulation body maximizes the distance between the two lines, whereby their own insulation is thereby preferably reduced to an acceptable minimum. The insulation thickness of the lines and the insulation material are thereby preferably selected so that sufficient safety against dielectric breakdown is provided.

The insulation body arranged additionally between the lines in the central opening of the current sensor thereby increases the distance between the two lines, particularly the distance of their two conductors from one another, and thus reduces the capacitive leakage current as well as potential partial discharges. Thus, electromagnetic disturbances originating from partial discharges can be reduced and the accuracy and safety of the current detection can be increased. Particularly the time resolution of the current sensor can be increased, because short-term fluctuations of the measurements, that is measurement noise, can be reduced. The current sensor remains functional in unlimited manner even under adverse environmental conditions, such as reduced air pressure in altitudes of 3,000 m to 5,000 m above sea level and/or increased air humidity. The insulation body minimizes partial discharge in the area of the current sensor during measurement of RF currents (250 kHz up to 2.5 MHz). The additional insulation body increases the distance between the two lines while only limited space is provided, particularly inside the opening of the current sensor.

Non-polar insulation material is preferably used as insulation material for the current sensor, such as PE, PP, PTFE or PI, which comprises a high specific volume resistance as well as minimum alternating voltage losses at high frequencies. Thus, it heats unremarkably and hardly allows current flow. Due to this, an interior partial discharge is highly suppressed. Preferably the insulation body is thereby at least slightly elastically configured in order to allow a simple handling and a local fixation of the two lines inside the opening.

For the insulation of the lines as well as for the insulation of the insulation body, preferably a material with high dielectric strength is used (for example silicone or PTFE). Thereby also the dielectric strength of the two lines against adjacent potentials outside the sensor is guaranteed.

The insulation body arranged in the current sensor between the two lines inside the opening acts as spacer, which brings the two lines passing through the opening to a maximum distance. Basically, the insulation body can consist of inorganic material, such as ceramic, which does not allow partial discharges and separates the two lines in terms of partial discharges optimally. However, as mentioned above, also nonpolar materials can be used, such as PTFE, nylon, etc., which only react in minor manner to alternating voltages and largely suppress partial discharge due to the low current flowing in the insulation.

The two lines respectively comprise a central electrical conductor in form of a single wire or a wire bundle (strand), which is coated by an electrically insulating coating. The coatings of the lines and the insulation body can consist from identical or different materials, particularly non-polar plastics. Amongst others each of the above-listed plastics can be used.

The coatings of the lines preferably comprise a circular cross-section respectively, in the center of which the respective conductor is arranged. The sum of the diameters of the two coatings is preferably smaller than the width of the opening. The insulation body arranged between the lines comprises such a thickness that it fits just sufficiently between the two lines without or with low mechanical tension and brings the lines into contact with the inside of the opening wall.

Preferably the insulation body is configured elastically so that its thickness can be compressed. For this purpose, it can comprise two legs that are configured to be shifted elastically or resiliently toward and away from each other. However, it can also be configured as an entirely deformable compact body. On its surfaces facing the lines it can be configured in concave manner in order to locally fixate the lines.

In an embodiment the surfaces facing the lines limit jaw-like cavities that abut against the line along more than half of the circumference of the line arranged therein. In doing so, a substantially homogeneous insulation is provided between the two lines, which counteracts partial discharges. Additionally on the surfaces of the insulation body abutting against the lines a conductive layer can be provided, for example a metallization. The equipotential surface created in this manner counteracts a local concentration of the electrical field and thus the creation of partial discharges. Conductive areas can be provided also at other locations. For example, a conductive layer can be arranged in the center or symmetry plane between the lines. In doing so, a symmetric balance of the electrical field can be achieved.

The opening of the sensor can be circular. For example the magnetic circuit can be realized by a hollow cylindrical part or as a part configured as torus. Other forms are however possible, particularly the openings as well as the outer circumference of the sensor can be limited in polygonal manner. Further, it is possible to provide the magnetic circuit with an opening divided by the insulation body. The insulation body can be mechanically connected with the magnetic circuit. For example, it can be connected with the magnetic circuit by means of a latched connection, a clamping connection, an adhesive connection or by any other appropriate means.

In a preferred embodiment the insulation body is configured as a substantially planar triple-leg part in which two outer tongues are provided that extend around the sensor on the outer side. From a joke connecting the two tongues, an inner tongue extends parallel to the outer tongues, so that it can be inserted into the opening of the sensor. This insulation body can be inserted in the opening of the magnetic circuit prior to passing the lines through the sensor, wherein the insulation body then remains on the sensor during and after the installation of the lines. Preferably, the free ends of the two outer tongues are provided with projections (noses) extending inwardly, by means of which the insulation body can be held on the sensor in a manner avoiding unintentional release.

### BRIEF DESCRIPTION OF THE DRAWINGS

Further details of advantageous embodiments are subject matter of dependent claims as well as the drawing and the associated description. The drawings show:

FIG. 1 an apparatus with a connected monopolar instrument that acts on biological tissue, which is connected with the apparatus via a neutral electrode, in a schematic illustration,

FIG. 2 the apparatus according to FIG. 1 during supply of a bipolar instrument,

FIG. 3 a current sensor with torus-shaped magnetic circuit in perspective illustration,

FIGS. 4 to 8 the current sensor with different insulation bodies in schematic front view respectively,

FIG. 9 the current sensor according to FIG. 8 in a perspective illustration and

FIG. 10 the insulation body of the current sensor according to FIG. 9 in a schematic perspective illustration.

### DETAILED DESCRIPTION

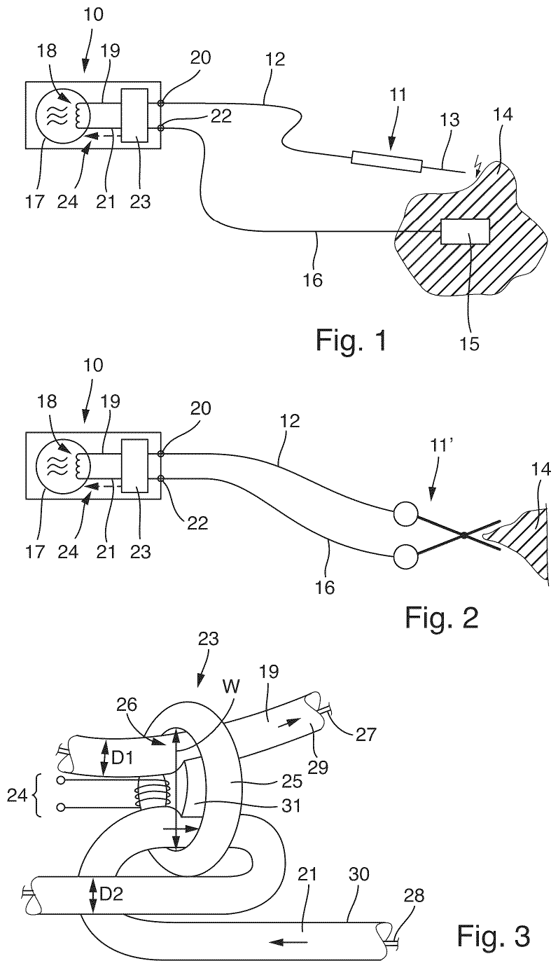

In FIG. 1 an apparatus 10 is illustrated with an instrument 11 connected thereto, wherein the instrument 11 is connected with the apparatus 10 by means of a cable 12. The instrument 11 comprises an electrode 13 for influencing biological tissue 14 on which a neutral electrode 15 is attached. Therefrom current introduced by means of the electrode 13 into the tissue 14 is guided back to the apparatus 10 by means of a cable 16.

The apparatus 10 comprises a generator 17 for providing a high-frequency voltage, for example up to 5 kVp or even more at a frequency between 250 kHz and 2.5 MHz. This generator 17 comprises an output circuit 18, which is connected via a first line 19 with a first apparatus output 20 and via a second line 21 with a second apparatus output 22. Also higher voltages and/or other frequencies are possible.

FIG. 2 illustrates the same apparatus 10 during supply of a bipolar instrument 11′, for example during cutting or during coagulation of tissue 14. Again, the instrument 11′ is connected via two cables 12, 16 with the apparatus outputs 20, 22, wherein the cables 12, 16 can also be combined in form of a two-or multi-core cable.

In the apparatus 10 the two lines 19, 21 pass through a current sensor 23, which is configured to detect the current supplied to the instrument 11 and to produce a measurement signal 24 supplied to generator 17. The measurement signal can be an electrical or another signal (for example optical signal) and can be used for controlling generator 17.

Between the two apparatus outputs 20, 22 and thus between the lines 19, 21 a voltage between some 100 Vp and multiple kVp (kilovolt peak voltage) applies depending on the respective treatment method. The two lines 19, 21 are thereby individually illustrated in FIG. 3 together with current sensor 23. The latter compresses a magnet circuit 25 having a central opening 26 through which the two lines 19, 21 pass. For example, the magnet circuit 25 can be configured as torus-shaped or hollow cylindrically shaped body, for example made of low-loss magnetic ferrite or the like.

A sensor coil can be wound on magnetic circuit 25 providing the measurement signal 24 at its ends. The RF alternating current flowing through lines 19, 21 creates an alternating magnetic field in the magnetic circuit that induces the measurement signal 24 in the sensor coil, for example as voltage signal. Noise components, which can result from sporadic charge movements in the vicinity of the measurement coil, may superimpose the measurement signal 24. Such charge displacements can result from partial discharges within electrically non-conductive elements or objects in the proximity of the measurement coil.

The two lines 19, 21 are both guided through opening 26 so that the current flows in the same sense through the opening 26 as indicated by arrows, in order to measure the sum of the current supplied to the instrument 11, 11′ and the current coming back therefrom. The two lines 19, 20 thereby comprise a conductor 27, 28 coated by means of an insulating coating 29, 30. The respective conductor 27, 28 can be realized by means of a wire or also a wire bundle, for example a strand. The coating 29 preferably comprises a circular cross-section and consists of a plastic having low dielectric losses, such as polyethylene, polytetrafluoroethylene or others.

The insulation thickness of the coatings 29, 30 is dimensioned so that the conductors 27, 28 have a dielectric strength even under the maximum considered voltages (for example 5 kVp). It shows that also in case of coatings 29, 30 with maximum thickness so that the two lines just fit through the opening 26, charge displacements can occur in the coatings 29, 30 which are harmless with regard to the insulation, however appear as disturbing currents that falsify the measurement signal 24. This particularly applies during detection of only low currents in the lines 19, 20 as they occur during working on tissue that is high ohmic (or has become high ohmic). The disturbance signal introduction can falsify the measurement signal 25 in the worst case up to unrecognizable condition, particularly if the measurement signal itself is to be zero. In addition, due to such partial discharges, a distortion of the waveform of the measurement signal 24 and thus wrong interpretations, for example about the tissue condition, would be possible. The partial discharges can superimpose a noise signal onto the measurement signal resulting in wrong measurements.

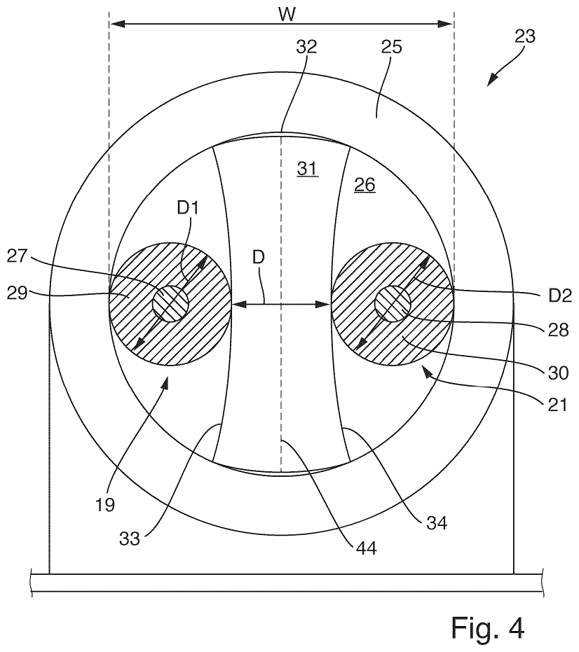

In the apparatus 10 and the current sensor 23, according to the invention, the coatings 29, 30 are however remarkably thinner. They are particularly so thin that they can be easily and with play inserted through the opening 26, which can have a width of only 5 mm, for example. Thus, the sum of the diameters D1 and D2 of the two lines 19, 20 is less than the width W. The distance between the lines 19, 21 inside the opening 26 is maximized by means of an insulation body 31 that is arranged between the lines 19, 21 and urges them away from one another against the inner side of the magnetic circuit 25 and thus against the wall of the opening 26. The sum of the thickness D of the insulation body and the diameters D1 and D2 corresponds therefore to the width Was particularly apparent from FIG. 4.

Due to maximizing the distance of the conductors 27, 28 from one another inside the opening 26 and the arrangement of the insulation body 31 between the lines 19, 21, partial discharges in the coatings 29, 30 and thus in the current sensor 23 are avoided. It involves partial discharges that disturb the detection of the current in the lines 19, 21 and could in this manner fake sparks or in fact non-existing tissue reactions or tissue characteristics. Due to suppressing partial discharges within the current sensor 23 and the accompanying lower disturbance of measurement signal 24 an improved analysis of the detected currents including their temporal progresses is made possible, so that improved conclusions can be made on the operation of the instrument 11, 11′ as well as the condition of the tissue 14 as well as the change thereof.

The above-mentioned explanations apply without limit also for the subsequent description of modified embodiments of the invention:

First, it has to be noted that the current sensor 23 according to FIG. 3 is connected in the form of a sum current sensor. However, it could also be configured as differential current sensor in that one of the lines 19 or 21 passes the opening 26 in a direction opposite to the direction illustrated in FIG. 3. Also here, the entire voltage difference is applied between the two lines 19, 21, wherein however only a small difference between the two currents in the lines 19, 21 is detected, which is created by capacitive leakage currents, for example. Specifically in this case, however, the avoidance of partial discharge and thus the avoidance of disturbances of the signal is particularly useful.

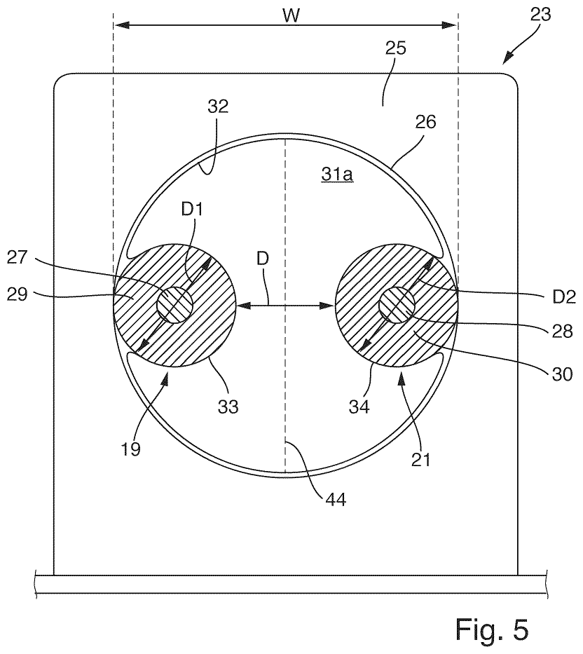

The magnetic circuit 25 can also be hollow cylindrically, as illustrated in FIG. 4, different to the torus shape depicted in FIG. 3. Further, it can have a polygonal shape on the inner side and/or the outer side, as illustrated in FIGS. 5 to 9. The insulation bodies 31a, 31b, 31c, 31d illustrated in FIGS. 5 to 9 can also be used in the magnetic circuits 25 according to FIG. 3 or 4.

The insulation body 31a according to FIG. 5 comprises an outer surface 32 on its outer circumference, which matches with the wall of the opening 26 and, for example, follows a cylinder. The surfaces 33, 34 facing the lines 19, 21 are configured in concave manner in the insulation body 31 according to FIG. 4 as well as in the insulation body 31a according to FIG. 5. In the embodiment according to FIG. 5 the surfaces 33, 34 enclose the lines 19, 21 and are in contact therewith. Both insulating bodies 31, 31a can be rigid, for example consist of a mineral material, such as ceramic, or can be made of plastic alternatively. In the latter case they can be configured in a slightly compressible manner in order to guarantee a strong seating connection inside the opening 26.

It is possible to provide the surfaces 33, 34 with an electrically conductive layer. The latter forms an equipotential surface avoiding or at least strongly reduces a local concentration of the electrical field. Alternatively or additionally, in a center plane located symmetrically between the two conductors 27, 28 an electrically conductive layer 44 can be provided. The latter also contributes to homogenization of the electrical field.

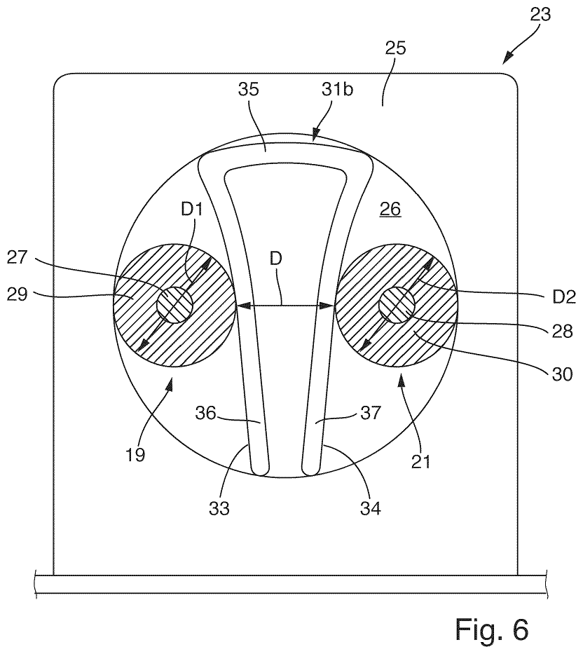

A modified embodiment of the insulating body is shown in FIG. 6 in form of insulating body 31b. Insulating body 31b comprises two legs 36, 37 that extend away from a common back section 35, wherein the legs 36, 37 are made of insulation material and urges the lines 19, 21 away from one another. The legs 36, 37 are spring elastically arranged and configured. The insulation body 31b can be configured as U-shaped plastic profile. The legs 36, 37 of insulation body 31b are only slightly curved. Alternatively, they can also be configured to tightly extend along the outer side of lines 19, 20. In the latter case, it can be expedient to provide a metallization on the surfaces 33, 34.

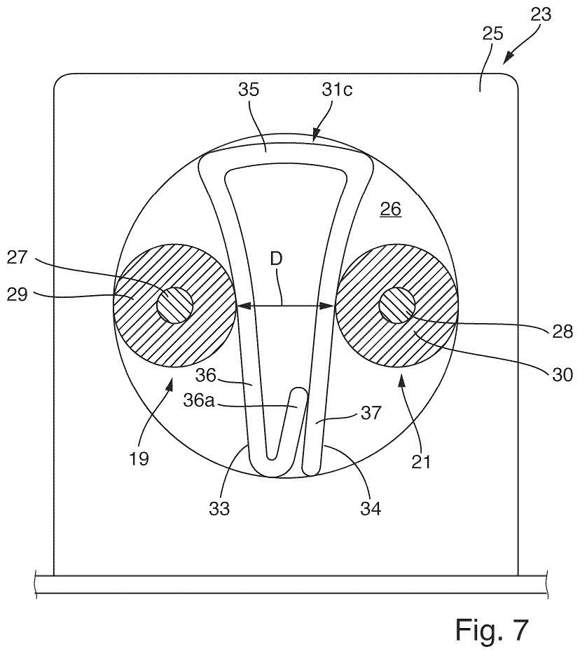

In doing so, it is also possible that the legs 36, 37 are not only connected with each other by means of the back section 35 as illustrated in FIG. 6. Rather the insulation body 31c can be configured according to FIG. 7. In this configuration an additional spring tension is created by means of an inwardly angled section 36a of one of the legs 36, 37 which is supported on the respective upper leg (here: leg 37).

All insulation bodies 31, 31a, 31b, 31c can have the same axial length (orthogonal to the drawing plane in FIGS. 4 to 7) as the opening 26. Preferably, however, they are configured to be slightly longer in order to avoid partial discharges between the lines 19, 21 not only inside the opening 26, but also in the proximity thereto.

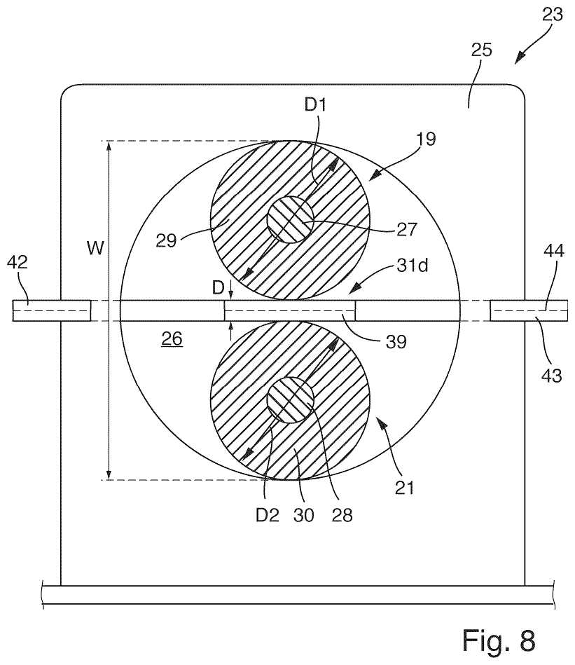

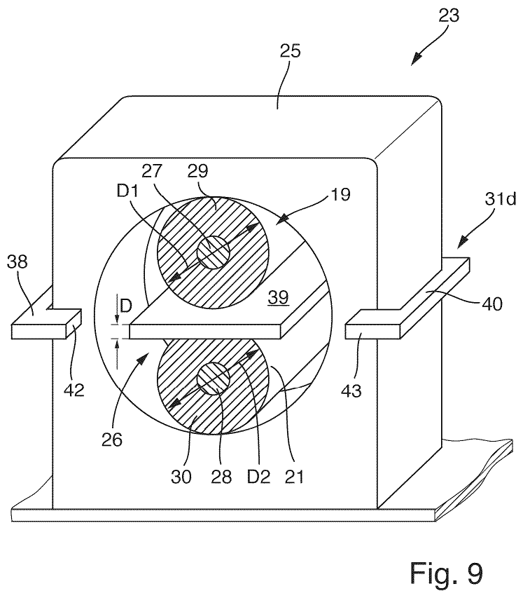

Another particularly easy-to-handle and also effective insulation body embodiment is the insulation body 31d according to FIGS. 8 to 10. It is preferably made of flexible flat material, for example cut from a flat flexible plastic material and comprises three strip-shaped tongues 38, 39, 40 arranged in a common plane, which extend parallel to one another away from a back section 41. The two outer tongues 38, 40 can have inwardly extending sections 42, 43 at their free ends, wherein the sections 42, 43 are suitable to grippingly extend along the magnetic circuit 25, as shown in FIG. 9, while the center tongue 39 is located between the lines 19, 21.

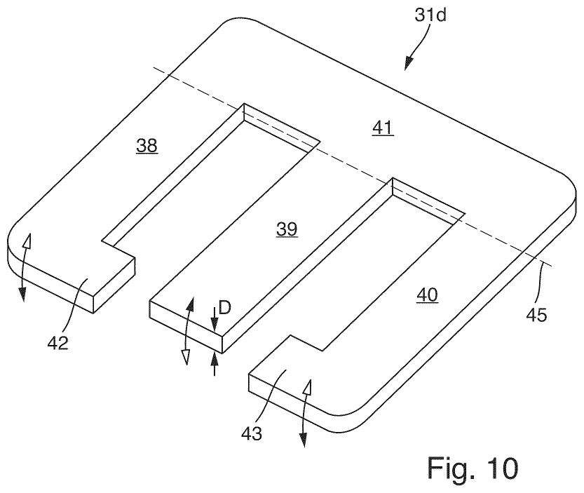

The center tongue 39 and preferably also the outer tongues 38, 40 are thereby held on the back section 41, preferably in a flexible manner. The center tongue 39 can be moved out of the plane defined by the tongues 38, 40. The outer tongues 38, 40 can be deflected in opposite direction, as indicated by (black and white colored) arrows in FIG. 10. The tongues 38, 39, 40 can thereby pivot around a pivot axis 45 located inside the back section 41 or in its proximity, whereby the tongues 38, 39, 40 can also be flexibly curved themselves. The pivot axis 45 marks the center of a connection area of the insulation body 31d, which is in so far flexible.

The insulation body 31d can be connected to the magnetic circuit 25 prior to the installation of the lines 19, 21, in that the tongues 38, 39, 40 are spread apart by means of torsion in opposite direction, so that the center tongue 39 can be inserted into the opening 26. After release all tongues 38, 39, 40 pivot back into one plane, wherein the outer tongues 38, 40 grippingly extend around magnetic circuit 25. The center tongue 39 can thereby have a width equal to the width of the opening 26 or can also be slightly narrower. In any case it separates the two lines 19, 21. The tongue 39 has a thickness D that in sum together with diameters D1, D2 of lines 19, 21 is equal to the width W of opening 26.

For each of the insulation bodies 31, 31a to 31d it applies that it can comprise one or more conductive coatings 44. In the insulation bodies 31, 31a, 31d these layers are preferably arranged in a center plane between the lines 19, 21 and thus define an equipotential plane. The latter can contribute to avoid local field inhomogeneities and thus also to reduce partial discharges. It is also possible to provide the surfaces 33, 34 with a conductive coating respectively.

According to the invention a ring-shaped magnetic circuit 25 is provided for a current sensor 23 of an apparatus 10 for supplying an instrument 11, 11′, wherein two lines 19, 21 supplying the instrument 11 and a neutral electrode 15 or the instrument 11′ pass through the magnetic circuit. The coatings 29, 30 of the two lines 19, 21 comprise sufficient thickness dimensioned with regard to their dielectric strength to the voltage load of lines 19, 21. The thickness is however limited so that an insulation body 31 fits between the two lines 19, 21 consisting of a nonpolar insulation material with high insulation capabilities, similar to the coatings 28, 30, for example consisting of a plastic with low loss angle. The insulation body 31, 31a to 31d reduces partial discharges in the area of the current sensor 23, which would otherwise result in electromagnetic emissions and thus distortions of the signals 24 created by the current sensor 23.

### REFERENCE SIGNS

10 apparatus
11 instrument
12 cable
13 electrode
14 tissue
15 neutral electrode
16 cable
17 Generator
18 output circuit
19 first line
20 first apparatus output
21 second line
22 second apparatus output
23 current sensor
24 measurement signal
25 magnetic circuit
26 opening
27 first conductor
28 second conductor
29 first coating
30 second coating
31 insulation body
32 outer surface
33, 34 surfaces
35 back section
36, 37 leg of insulating body 31b or 31c
36a projection
38-40 leg of insulating body 31d
41 back section of insulating body 31d
42, 43 projections
44 electrically conductive layer
45 pivot axis

1. An apparatus (10) for supply of an electrosurgical instrument (11, 11′) with a treatment current, the apparatus comprising:
a generator (17) comprising an output circuit which is connected with a first apparatus output (20) via a first line (19) for supply of the electrosurgical instrument (11, 11′) and which is connected to a second apparatus output (22) via a second line (21) for guiding a current back;
a current sensor (23) for detection of a sum or difference of a current flowing in the first line (19) and a current flowing in the second line (21);
wherein the current sensor (23) comprises a magnet circuit (25) having a central opening (26) through which the first line (19) and the second line (21) pass; and
an insulation body (31, 31a-31d) arranged in the central opening (26) between the first and second lines (19, 21).

2. The apparatus according to claim 1, wherein the first and second lines (19, 21) each comprise a central electrical conductor (27, 28) and an electrically insulating coating (29, 30) that coats the central electrical conductor (27, 28).

3. The apparatus according to claim 2, wherein each of the electrically insulating coatings (29, 30) has a circular cross-section, wherein the respective central electrical conductor (27, 28) is arranged centrally in the circular cross-section.

4. The apparatus according to claim 2 wherein each electrically insulating coating (29, 30) comprises a diameter (D1, D2) and the central opening (26) comprises a width (W), wherein the sum of the diameters (D1, D2) of the electrically insulating coatings is less than the width (W) of the central opening.

5. The apparatus according to claim 4, wherein the insulation body (31, 31a-31d) has a thickness (D), wherein a sum of the diameters (D1, D2) of the electrically insulating coatings and the thickness of the insulation body is equal to the width (W) of the central opening.

6. The apparatus according to claim 4, that wherein the insulation body (31, 31a-31d) comprises a thickness (D), wherein the sum of the diameters (D1, D2) of the electrically insulating coatings and the thickness of the insulation body is larger than the width (W) of the central opening.

7. The apparatus according to claim 1, wherein the insulation body (31, 31a-31d) is elastic.

8. The apparatus according to claim 1, wherein the insulation body (31, 31a-31d) comprises a thickness (D), wherein insulation body (31, 31a-31c) is compressible in a direction of in its thickness (D).

9. The apparatus according to claim 1, wherein the insulation body (31b, 31c) comprises two legs (36, 37) that are elastically shiftable away from and toward one another.

10. The apparatus according to claim 1, wherein the insulation body (31, 31a) comprises concavely curved surfaces (33, 34) facing the first and second lines (19, 21), respectively.

11. The apparatus according to claim 2, wherein the central opening (26) is configured in a circular-shaped manner.

12. The apparatus according to claim 1, wherein the insulation body (31a) has an outer shape adapted to the central opening (26) and comprises two surfaces (33, 34) adapted respectively to the first and second lines (19, 21), wherein the two surfaces (33, 34) each delimit a jaw-like cavity.

13. The apparatus according to claim 1, wherein the insulation body (31d) comprises a holding section (41;38,42;40,43) extending around the magnetic circuit (25).

14. The apparatus according to claim 13, wherein the insulation body (31d) comprises a tongue (39) extending away from a back section (41) of the holding section (41;38,42;40,43) and extending through the central opening (26).

15. The apparatus according to claim 14, wherein the tongue (39) and the holding section (41;38,42;40,43) are configured as a monolithic flat part, wherein the tongue (39) and the holding section (41;38,42;40,43) are pivotable relative to one another.
